(19) United States
(12) Patent Application Publication
(10) Pub. No.: US 2010/0298282 A1
(43) Pub. Date: Nov. 25, 2010

(54) Treatment of Ocular Conditions and the Side-Effects of Glucocorticoids

(76) Inventors: Alan Geoffrey Roach, Oxon (GB); Paul Goldsmith, Oxon (GB); Ruth Margaret Barrett, Oxon (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/161,413

(22) PCT Filed: Jan. 19, 2007
(86) PCT No.: PCT/GB07/00191

§ 371 Date: Aug. 9, 2010

(30) Foreign Application Priority Data
Jan. 19, 2006 (GB) 0601092.0

(51) Int. Cl. A61K 31/575; A61P 27/06; A61P 27/12; A61P 9/12; A61P 27/02

(52) U.S. Cl. 514/179

(57) ABSTRACT

A compound having glucocorticoid receptor (GR) antagonist activity and a clogP value of less than 5 is useful for the treatment of an ocular condition. Further, a compound having glucocorticoid receptor (GR) antagonist activity is useful, for the treatment of a patient exhibiting side-effects of the administration of a glucocorticosteroid, e.g. wherein the glucocorticosteroid and said compound are administered by different routes.

### FIELD OF THE INVENTION

This invention relates to the treatment and prophylactic prevention of ocular conditions and the side-effects resulting from the taking of glucocorticoid medicaments (steroids), primarily by the inhaled route, topically to skin or orally.

### BACKGROUND OF THE INVENTION

Steroids are extremely powerful and useful drugs widely prescribed in medicine for a variety of different indications, in particular conditions with a significant inflammatory component such as asthma, COPD, rheumatoid arthritis, psoriasis, ulcerative colitis and Crohn's disease. Unfortunately, steroids have many serious side-effects, severely limiting their usefulness. Such side-effects include gastrointestinal (e.g. dyspepsia, peptic ulceration, abdominal distension, acute pancreatitis, oesophageal ulceration and candidiasis), musculoskeletal (e.g. myopathy, osteoporosis, avascular necrosis and tendon rupture), endocrine (e.g. adrenal suppression, diabetes, Cushing's syndrome, weight gain, increased appetite, susceptibility to infection, menstrual irregularities, hirsutism), neuropsychiatric (e.g. euphoria, depression, insomnia, psychosis), ophthalmic (e.g. cataract, intraocular hypertension and glaucoma, corneal and scleral thinning), dermal (e.g. skin atrophy, easy bruising, poor wound healing, acne, striae, telangiectasiae) and other effects (e.g. hypertension, fluid retention, growth retardation).

These side-effects are commonplace with systemic exposure after giving steroids orally. However, these side-effects are also experienced when steroids are administered in high doses topically to the lung via the inhaled route to patients with severe asthma. Systemic side-effects are also noted after high doses of steroids are given topically to the eye or the skin, although ocular side-effects are significant with steroids applied topically on or implanted into the eye, and skin-thinning is a problem associated with dermal application of steroids.

The effective treatment of steroid-induced cataract and other ocular conditions is problematical. For example, current treatment for cataracts is largely limited to their surgical removal. This radical procedure is only conducted once the cataract has progressed to a significant extent and therefore the patient will already have suffered with a period of poor vision. Furthermore, the operation is costly and has risks particularly in the elderly patient population. Additionally, the dynamic control of accommodation is lost with the insertion of an artificial lens, so the overall quality of vision is not as good as in a patient with completely normal eyes. Steroid-induced glaucoma and raised intraocular pressure are treated either surgically or with general anti-glaucoma medications including prostanoid analogues, beta-adrenoceptor blockers, miotics, sympathomimetics and carbonic anhydrase inhibitors, either alone or in combination. Despite these various therapeutic options, the use of some treatments is limited (e.g. use of beta-blockers in asthmatics taking steroids may exacerbate asthmatic symptoms) and steroid-induced intraocular hypertension/glaucoma is relatively resistant to these medicaments.

Mifepristone (RU486) is used clinically as “the morning after pill” and provokes chemically induced abortions. Mifepristone possesses both progesterone (PR) and glucocorticoid receptor (GR) antagonist properties.

The clinical use of mifepristone is contraindicated in patients being treated chronically with glucocorticoids, as its GR antagonist properties would compromise the anti-inflammatory efficacy of the steroid treatment. In addition, mifepristone is contraindicated in severe asthmatics (a condition treated with inhaled or oral steroids) (Exelgyn Laboratories Information for Mifegyne (mifepristone) patient information leaflet: see http://www.emc.medicines.org.uk/).

Due to its highly lipophilic nature, mifepristone has been formulated as a 1% suspension in hydroxypropyl methylcellulose eyedrops and administered topically (as a milky suspension) to the eyes of rabbits, and falls in intraocular pressure have been noted, after 4-13 weeks of treatment (Phillips et al., 1984, Lancet, 1, 767-768; Green et al., 1985, Curr. Eye Res., 4, 605-612).

The levels of mifepristone in various parts of the eyes of rabbits, after administering mifepristone as a 1% suspension (in hydroxypropyl methylcellulose) topically to the eyes, have been measured. They ranged from about 5 μM at 1 hour, 0.5 μM at 8 hours and 0.05 μM at 24 hours (Cheeks and Green, 1986, Curr. Eye Res., 5, 705-709). Based on the potency of mifepristone at GR receptors, these data indicated that significant GR antagonism occurred in the eye for approximately 8 hours after topical application to the eye of rabbits. In addition, mifepristone has been injected subconjunctivally as a 1% suspension to rabbits, and again falls in intraocular pressure were reported (Tsukahara et al., 1986, Br. J. Opthalmol., 70, 451-455). Mifepristone has been shown to bind to GR receptors in the iris-ciliary body of the rabbit eye and specifically blocks the binding of the GR agonist triamcinolone (Munden & Schmidt, 1991, Arch. Opthalmol., 110, 703-705).

It has been shown that topical application of a steroid to rat skin reduced skin thickness. Mifepristone given either subcutaneously or topically to the skin blocked atrophogenic effect of the topical steroid without affecting its anti-inflammatory effect in rat skin (Iwasaki et al., 1995, J. Dermatol. Sci., 10, 151-158).

Three metabolites of mifepristone generated in humans have been reported to possess activities at both GR and PRs (Deraedt ° tel., 1984, Pharmacokinetics of RU486, in “The Antiprogestin Steroid RU486 and Human Fertility Control”, ed. Baulieu & Segal. pp 103-122, Plenum Press, New York). These are the demethylated metabolite RU42633, the didemethylated metabolite RU42848 and the hydroxylated metabolite RU42698.

Many selective GR antagonists are known, and some are in development. Some such compounds are disclosed in WO03043640, WO00116128, WO02064550, WO04000869, WO00147859, WO00244120, WO05070893, WO05087769, WO00066522, Morgan et al., 2002, J. Med. Chem., 45, 2417-2424, Akritopoulou-Zanze et al., 2004, Bioorg. Med. Chem. Lett., 14, 2079-2082 (compound 19c, also known as A-362947) and Mohler et al, 2007, Expert Opin. Ther. Patents, 17, 59-81. GR antagonists are proposed to be useful in therapeutic areas such as anxiety disorder, pychosis, drug dependence, Cushing's disease, dementia, major depressive disorder, diabetes mellitus, obesity, hyperlipidaemia and hypertension.

### SUMMARY OF THE INVENTION

The present invention is based at least in part on the realisation that the side-effects observed with steroid therapy in tissues or organs that are remote or distinct from the tissues requiring treatment by the steroid can be prevented or reduced, by the use of a compound possessing GR antagonist activity given at such doses, either locally to a remote site (i.e. a site distinct to that being treated with the steroid), or systemically. Significant levels of steroids are absorbed into the systemic circulation and tissues after they are given topically in high concentrations for a protracted period. Thus, in this latter scenario, the GR antagonist given orally can achieve sufficiently pharmacologically active GR antagonist concentrations at sites that are peripherally remote from those that are targeted by locally by the topically applied steroid to block steroid-induced side-effects in these tissues whilst not achieving sufficiently high enough levels to impact on the level of anti-inflammatory activity within the target tissue where the steroid is applied.

The topical sites where steroids are applied may be by inhalation to the lung and administration to the eyes and skin. The route of administration of the GR antagonist may be systemic administration, ocular or dermal. However, in the latter two cases, the GR antagonists would not be administered by this route if the steroid was also being given by that same route, unless it was specifically desired to reverse an unexpected or undesired side-effect of prior topical administration of steroid.

A particular aspect of the invention is the treatment of ocular disorders. According to this aspect a compound having glucocorticoid receptor (GR) antagonist activity and a clogP value of less than 5 is used for the treatment of an ocular condition.

Other aspects and embodiments of the invention include:
(i) Orally administered GR antagonist versus systemic side-effects produced by inhaled steroids.
(ii) Orally administered GR antagonist versus side-effects produced by steroids applied dermally, i.e. preferably topically to the skin or into the dermis.
(iii) Topical (ophthalmic or dermal) GR antagonist versus the side-effects produced by oral steroids.
(iv) Topical (ophthalmic) GR antagonist versus side-effects produced by inhaled steroids.
(v) Topical (ophthalmic) GR antagonist versus side-effects produced by steroids applied dermally.
(vi) Topical (dermal) GR antagonist versus side-effects produced by inhaled steroids.

The side-effects may be the consequence of endogenous or exogenous steroid. If endogenous the steroid levels may be abnormally raised, as in Cushing's disease, or within the normal range but with the agonist effects of the steroid contributing to a physiological or pathological effect, such as the control of intraocular pressure. If exogenous, the compound used in the present invention is administered by a route different from that used to administer the steroid.

### DESCRIPTION OF THE INVENTION

Depending on the intended use and route of administration, various criteria may be used to determine a GR antagonist for use in the invention. For example, a compound that may be used for the treatment of steroid-induced side-effects (in particular ocular, osteo and dermal effects), is one in which the GR antagonist activity is at least 1% that of mifepristone as determined in the in vitro GR binding assay described in Gill et al, 1986, J. Med. Chem. 29, 1537-1540, and Morgan et al, 2002, J. Med. Chem. 45, 2417-2424, and/or the in vitro functional assay described in WO2005/087769. The GR antagonism preferably negates more than 30%, more preferably more than 50%, and most preferably more than 90%, of the GR agonism at sites distant from the site of administration of the corticosteroid, and negates less than 70%, more preferably less than 50%, and most preferably less than 10%, of the GR agonism at the site of intended action of the corticosteroid. In the in vitro functional assay described above, and also below in Example 1, the preferred range of activity is 0.1 nM to 10 μM (the Ki activity of mifepristone by this test is approx. 0.5 nM).

For ophthalmic use in particular, the active compound should be less lipophilic than mifepristone. Lipophilicity can be readily determined, using available methods, by one of ordinary skill in the art. For example, clogP may be calculated using the Interactive Analysis LogP predictor website www.lociP.com http://www.logp.com/. The value is preferably no more than 5, more preferably no more than 4.5 and most preferably no more than 4.

Compounds that satisfy the criteria for use in the invention are generally described in the given prior art (incorporated herein by reference). More specifically, they include three known metabolites of mifepristone, (i.e. the monodemethylated and didemethylated derivatives (RU42633 and RU42848) and the alcoholic non-demethylated analogue (RU42689)). Another is Compound A-362947, i.e N-[1-methoxy-6-(3-methylphenyl)-6H-benzo{c}chromen-8-yl]methanesulfonamide, which is a representative selective GR antagonist. Further such compounds are Org-34850, i.e. 11-(4-dimethylaminophenyl)-17-hydroxy-17-(4-methanesulfonylphenylethynyl)-13-methyl-1,2,6,7,8,11,12,13,14,15,16,17-dodecahydrocyclopenta[a]phenanthren-3-one, and Org-34517, i.e. 11-benzo[1,3]dioxol-5-yl-17-hydroxy-13-methyl-17-prop-1-ynyl-1,2,6,7,8,11,12,13,14,15,16,17-dodecahydro-cyclopenta[a]phenanthren-3-one

The invention can be used for the treatment and prevention of steroid-induced side-effects such as cataracts, glaucoma (including idiopathic glaucoma) or intraocular hypertension, osteoporosis and skin thinning. An enhanced effect on steroid-induced raised intraocular pressure may be achieved by the co-administration of a GR antagonist with either a prostanoid (e.g. latanoprost, bimatoprost, travoprost or unoprostone), β-adrenoceptor antagonist (e.g. timolol or betaxolol), carbonic anhydrase inhibitor (e.g. brinzolamide or dorzolamide) or α2-adrenoceptor agonist (e.g. apraclonidine or brimonidine).

In general, the active compound may be administered by known means, in any suitable formulation, by any suitable route. A compound of the invention is preferably administered orally or topically. In the case of preventing side-effects of inhaled steroids the GR antagonist can be delivered by such routes (oral, buccal, rectal) that provide sufficient levels systemically without impacting the therapeutic effects of the steroid within the lungs. The preferred topical routes are ophthalmic and dermal.

As indicated above, the condition to be treated may be caused by endogenous steroid production. Thus, the present invention may be used as a monotherapy to treat any condition described above. Alternatively, exogenous steroid may have been administered by known means, in any suitable formulation, by any suitable route. As indicated above, the steroid will typically have been inhaled or administered topically.

The compound is preferably formulated such that an equivalent or greater pharmacological effect to mifepristone is noted within the eye or the skin in relevant animal models. Such animals models used include reduction in intraocular pressure in rabbits (K. S. Lim et al., 2005, Invest. Opthalmol. Vis. Sci., 46, 2419-2423) and rat skin (K. Iwasaki et al., 1995, J. Dermatol. Sci., 10, 151-158).

The compositions may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.001-99% by weight of active compound. The compositions of the invention are generally prepared in unit dosage form. Preferably, a unit dose comprises the active ingredient in an amount of 0.001 to 500 mg. The excipients used in the preparation of these compositions are the excipients known in the art.

Appropriate dosage levels may be determined by any suitable method known to one skilled in the art. It will be understood, however, that the specific dose level for any particular patient will depend upon a variety of factors including the age, body weight, general health, sex, diet, time of administration, route of administration, rate of excretion, drug combination and the severity of the complaint. Preferably, the active compound is administered at a frequency of 1 to 4 times per day. A typical daily dosage is for the oral treatment is 5-500 mg daily and topically in formulations containing 0.001-10% active ingredient.

Compositions for oral administration include known pharmaceutical forms for such administration, for example tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions, and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch, gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, polyoxyethylene hydrogenated castor oil, fatty acids such as oleic acid, or in a mineral oil such as liquid paraffin or in other surfactants or detergents. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

The active compound may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for topical administration are also suitable for use in the invention. The compounds of the present invention may be administered by any means known to those skilled in the art for treatment of eye diseases. The compounds may be administered in a sterile preparation comprising the active compound or pharmaceutically acceptable salt thereof with a pharmaceutically acceptable vehicle or carrier therefore. The active GR antagonists disclosed herein may be administered to the eyes of a patient by any suitable means, but are preferably administered as a liquid or gel suspension of the active compound in the form of drops of liquid, liquid washes, sprays, ointments, or gel. Alternatively, the active compounds may be applied to the eye via liposomes or other carriers such as cyclodextrins. Further, the active compounds may be infused into the tear film via a pump-catheter system. Another embodiment of the present invention involves the active compound contained within a continuous or selective-release device, for example, membranes such as, but not limited to, those employed in the Ocusert System (Alza Corp., Palo Alto, Calif.). As an additional embodiment, the active compounds can be contained within, carried by, or attached to contact lenses, that are placed on the eye. Another embodiment of the present invention involves the active compound contained within a swab or sponge that can be applied to the ocular surface. Another embodiment of the present invention involves the active compound contained within a liquid spray that can be applied to the ocular surface. Another embodiment of the present invention involves an injection of the active compound directly into the lachrymal tissues or onto the eye surface, or intravitreal injection.

The topical solution containing the active compound may also contain a physiologically compatible vehicle, as those skilled in the ophthalmic art can select using conventional criteria. The vehicles may be selected from the known ophthalmic vehicles which include, but are not limited to, saline and aqueous electrolyte solutions, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride. In ophthalmic compositions, a chelating agent may be used to enhance preservative effectiveness. Suitable chelating agents are those known in the art, and, while not intending to be limiting, edetate (EDTA) salts like edetate disodium, edetate calcium disodium, edetate sodium, edetate trisodium, and edetate dipotassium are examples of useful chelating agents. It is understood that EDTA refers to a species having four carboxylic acid functional groups, and that these carboxylic acid groups may be protonated or deprotonated (i.e. in the salt form) depending upon the pH of the composition it is in. As is known in the art, buffers are commonly used to adjust the pH to a desirable range for ophthalmic use. Generally, a pH of around 5-8 is desired, however, this may need to be adjusted due to considerations such as the stability or solubility of the therapeutically active agent or other excipients. Antioxidants may be added to compositions of the present invention to protect the active compound from oxidation during storage. Examples of such antioxidants, but are not limited to, include vitamin E and analogs thereof, ascorbic acid and butylated hydroxytoluene (BHT).

Ophthalmic products are typically packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% weight/volume (“% w/v”).

A suitable formulation for administration to the eye comprises one or more of a cyclodextrin, methylcellulose and polyethylene glycol. Suitable respective amounts are 5-30%, 1-40% and 0.01-5%, e.g. 15%, 5% and 0.5%, respectively.

The pharmaceutically active compound may be dispersed in a pharmaceutically acceptable cream, ointment or gel. A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent. Topically administrable compositions may also comprise a matrix in which the active compound is dispersed, so that the compound is held in contact with the skin, in order to administer the compound transdermally.

The following Examples illustrate the invention.

### Example 1

The relevant activities of various compounds were tested. Results are given in Table 1.

TABLE 1

| | PR Binding | | GR Binding | | GR Antagonist | |
|---|---|---|---|---|---|---|
| | IC50 | Ki | IC50 | Ki | Mean IC50 | Mean Ki |
| Compound | (nM) | (nM) | (nM) | (nM)) | (nM) | (nM) |
| RU486 | 6.2 | 2.48 | 0.98 | 0.49 | 3.3 | 0.5 |
| RU42633 | 4.46 | 1.79 | 1.12 | 0.56 | 17.3 | 2.55 |
| RU42698 | 10.6 | 4.24 | 1.74 | 0.87 | 80.9 | 12.6 |
| RU42848 | 15.9 | 6.37 | 1.36 | 0.68 | 58.3 | 8.6 |
| A-362947 | 1327.4 | 530.9 | 5.64 | 2.82 | 363.3 | 50.4 |

GR binding was assessed using glucocorticoid radioligand binding, with the human recombinant receptor and [³H]-dexamethasone. The methods are described by Gill et al. and Morgan et al., supra.

PR binding was assessed using the same methods, using the human recombinant receptor and [³H]-progesterone. IC₅₀ values were determined from six-point concentration/inhibition curves performed in duplicate at semi-log concentrations.

The glucocorticoid reporter gene assay was performed in SW1353 human chondrosarcoma cell line that expresses native glucocorticoid receptors (Morgan et al., supra), stably transfected with a vector containing a glucocorticoid-responsive element (GRE) linked to the firefly luciferase gene. Treatment of the cells with dexamethasone induces expression of luciferase (EC₅₀^{dex} 10 nM). To test for GR antagonist activity, cells were incubated in 96-well plates for 24 hours with several dilutions of the compounds in the presence of 5xEC₅₀^{dex} (50 nM) dexamethasone and the inhibition of induced luciferase expression was detected by measurement of luminescence. For each assay, a concentration response curve for dexamethasone was prepared in order to determine the EC₅₀^{dex} required for calculating the Ki from the IC₅₀s of each tested compound. 10₅₀ values were determined from six-point concentration/inhibition curves performed in duplicate at semi-log concentrations. Compounds were tested on two separate occasions to determine inter-assay variability.

Kᵢ values were calculated from the equation

Ki=IC₅₀/(1+[Dex]/EC₅₀^{Dex})

Using the reference given above, clogP values are predicted, and are given in Table 2 (together with molecular weights).

TABLE 2

| Compound | Molecular Weight | CAS No. | cLogP prediction |
|---|---|---|---|
| RU486 | 429.6 | 84371-65-3 | 5.09 |
| RU42698 | 445.59 | 105012-15-5 | 4.21 |
| RU42633 | 415.57 | 104004-96-8 | 4.34 |
| RU42848 | 401.54 | 104004-92-4 | 3.57 |
| A-362947 | 395.47 | | 4.85 |
| Org-34850 | 569.75 | 162607-84-3 | 4.84 |
| Org-34517 | 430.54 | 189035-07-2 | 4.74 |

### Example 2

Healthy female New Zealand White rabbits were tested in a randomized, blind, controlled study, with 6 animals per treatment group. They were acclimatized for 7 days prior to the start of the study. Intraocular pressure (IOP) was measured in both eyes pre-treatment and at intervals after instillation of a single eye drop of vehicle or test compound into the left eye of each animal, administered from a dropper bottle. The volume of each drop was approximately 60-65 microlitres. The vehicle was a sterile solution of 15% hydroxpropyl-β-cyclodextrin, 5% polyethylene glycol, 0.5% hydroxypropylmethylcellulose in deionised water, adjusted to pH 7 with HCl or NaOH as appropriate. The test compounds RU486, RU42848 and RU42698 were dissolved at 3 mg/ml in the vehicle and RU 42848 was dissolved at 2 mg/ml in the vehicle. IOP was measured using a Mentor Tonopen XL (Lim et al., 2005, Invest Opthalmol Vis Sci 46 2419-23), after topical administration of 0.5% proxymetacaine HCl local anaesthetic. Ten recordings per eye per time point were taken and a mean reading documented.

The results are shown in Table 3. Compared to the vehicle-treated group, RU486 did not induce a statistically significant decrease in IOP at 3 hours (p=0.165) or 4 hours (p=0.337). RU42848 induced a significant decrease in IOP compared to vehicle at 3 hours (p=0.033) and 4 hours (p=0.019). RU42698 also induced a significant decrease compared to vehicle at 3 hours (p=0.039) and at 4 hours (p=0.045).

TABLE 3

| | IOP (mm Hg) at 3 hours | IOP (mm Hg) at 4 hours |
|---|---|---|
| Treatment | Mean (SEM) | Mean (SEM) |
| Vehicle | 13.83 (0.70) | 13.17 (0.48) |
| 0.3% RU486 | 11.67 (1.23) | 11.83 (1.19) |
| 0.3% RU42848 | 11.67 (0.49) * | 11.50 (0.34) * |
| 0.3% RU42698 | 11.83 (0.40) * | 11.83 (0.31) * |

1-21. (canceled)

22. A method for the treatment or prophylaxis of an ocular condition comprising administering an effective amount of the compound RU42698, or a pharmaceutically acceptable salt thereof.

23. The method of claim 22 wherein the ocular condition is intraocular hypertension.

24. The method of claim 22 wherein the ocular condition is cataract.

25. The method of claim 22 wherein the ocular condition is glaucoma.

26. The method of claim 25 wherein the ocular condition is idiopathic glaucoma.

27. The method of claim 22 wherein the ocular condition is induced by endogenous steroid production.

28. The method of claim 22 wherein the ocular condition is induced by exogenous steroid administration.

29. The method of claim 22 wherein the ocular condition is induced by exogenous steroid administration at a site distant from the eye.

30. The method of claim 22 wherein the ocular condition is induced by exogenous steroid administration which is topical to the skin, oral or by inhalation.

31. The method of claim 22 wherein the compound is administered topically to the eye.

32. The method of claim 22 wherein the compound is co-administered with: (a) a prostanoid; (b) a β-adrenoceptor antagonist; or (c) a carbonic anhydrase inhibitor.
